(19) United States
(12) Patent Application Publication
(10) Pub. No.: US 2024/0399004 A1
(43) Pub. Date: Dec. 5, 2024

(54) COMPOSITIONS AND METHODS FOR CLEANING AND STERILIZING SURGICAL INSTRUMENTS WHILE MITIGATING CORROSION

(71) Applicants: Kevin Rezzadeh, Los Angeles, CA (US); Niki Rezzadeh, Los Angeles, CA (US); Rudy Rezzadeh, Los Angeles, CA (US)

(72) Inventors: Kevin Rezzadeh, Los Angeles, CA (US); Niki Rezzadeh, Los Angeles, CA (US); Rudy Rezzadeh, Los Angeles, CA (US)

(21) Appl. No.: 18/678,165

(22) Filed: May 30, 2024

Related U.S. Application Data
(60) Provisional application No. 63/505,337, filed on May 31, 2023.

(51) Int. Cl. A61L 2/18

(52) U.S. Cl. CPC A61L 2/186; A61L 2202/15; A61L 2202/24

(57) ABSTRACT

Methods and compositions are contemplated which may be used to clean an instrument while minimizing the effects of corrosion upon these instruments. The composition may comprise an anticorrosive agent and an antiseptic. The combination of an anticorrosive agent of methylene blue and an antiseptic of chlorhexidine, hydrogen peroxide, povidone iodine, sodium hypochlorite, isopropyl alcohol, citric acid, or combinations thereof has been found to give effectual cleaning results upon contaminated instruments. The compositions and methods may be configured so as to achieve sterilization of the instrument. The compositions may further comprise additional components including an antibiotic, a tracer solution or tracer solutions, an adjuvant, and an indicator component.

### CROSS-REFERENCE TO RELATED APPLICATIONS

This application relates to and claims the benefit of U.S. Provisional Application No. 63/505,337 filed May 31, 2023, and entitled “COMPOSITIONS AND METHODS FOR STERILIZING SURGICAL INSTRUMENTS WHILE MITIGATING CORROSION,” the entire disclosure of which is hereby wholly incorporated by reference.

### STATEMENT RE: FEDERALLY SPONSORED RESEARCH/DEVELOPMENT

Not Applicable

### BACKGROUND

### 1. Technical Field

The present invention relates to methods and compositions for disinfecting and sterilizing medical instruments. More specifically, the present invention relates to methods and compositions for disinfecting and sterilizing medical instruments which minimize the effects of corrosion on those instruments.

### 2. Related Art

Various medical instruments are employed in surgical operations, many of which become contaminated with substances as a result of coming into contact with a patient and/or an unsterile surface. After being used in a step of a surgical operation and becoming contaminated via contact with the patient, these medical instruments may be cleaned to remove species such as soft tissue and body fluids like adhering blood. If the medical instrument is a smaller handheld instrument, there are a couple of procedures commonly performed during surgical operations to partially clean the instrument before it is used again in a later step of the same surgical operation (i.e., intraoperative usage).

One of these procedures, usually used with smaller handheld instruments, entails wiping down bloody/soiled instruments with a lab sponge (a cotton rag) before said instrument is used again in the same surgical operation. Such a procedure suffers from numerous drawbacks, one being its ineffectiveness at killing bacteria. While the bacteria residing in the soft tissue and blood removed by the sponge could be separated from the instrument with this procedure, any bacteria that has migrated or adhered to the instrument itself or debris that was not able to be removed mechanically may continue to occupy that instrument, creating a health risk of transferring bacteria from one bodily area of a patient to another when the medical instrument is next used. Additionally, this procedure may not be effective in treating certain portions of the medical instrument that are hard to reach, examples of which include the mouth of a rongeur, the hinge of a pair of scissors, and the grooves of a beveled grip or tip. Plus, this procedure is not always carried out in between steps of the surgical operation, as it can be difficult to carry out quickly in the midst of a surgical operation wherein a surgical technician may have to quickly transition from one surgical step to another.

An alternative cleaning procedure, usually suited for larger and more cannulated handheld medical instruments, may be performed by at least partially submerging a used instrument (preferably including the portion(s) of the instrument that came in contact with the patient or otherwise contains contamination) into a reservoir of a sterilized solution, usually referred to as a splash basin. However, this sterilized solution is typically just a simple solution of sterilized water, so while the splash basin procedure may initially be effective at treating the medical instrument and overcoming some of the deficiencies of the previous procedure described above, the reservoir may become progressively more contaminated over the course of a surgical operation with the blood, bacteria, and tissue introduced to the reservoir. The median rate of contamination for these splash basins is over 20% by the end of the surgical operation, but rates as high as 70% have been reported in some cases. On average, over forty instruments pass through these basins and are reused upon the operative wound of a patient, which can lead to a nidus of bacterial infection to materialize in the splash basin.

It may seem that a simple fix to this issue would be to introduce a sterilizing agent such as a surgical antiseptic to the reservoir in order to more effectively kill bacteria present in the reservoir's solution, but such a practice is rarely carried out; most surgical antiseptics that could theoretically be used unfortunately have corrosive properties that may affect the medical instruments they are applied upon. Many of these types of medical instruments are carefully constructed so that they may both carry out a successful operation and minimize the risk of damage and harm to the patient. Therefore, even minor cases of corrosion to a medical instrument could compromise its careful architecture and substantially increase this risk and/or make the instrument much less effective at its intended purpose. Thus, it is generally considered to be too risky to employ surgical antiseptic for intraoperative cleaning procedures, even if they could greatly reduce the risk of infection in between steps of the surgical operation. Antiseptics are instead typically applied directly to the operative wounds of the patient in order to prevent or at least partially treat infections, but without targeting the bacteria on the instrument, this practice can only go so far in preventing that bacteria from being introduced to the patient. It would be much more desirable to kill or remove these bacteria directly on the instrument.

Another intraoperative aspect that can be improved upon is the sterilization process of these surgical instruments. As known well by those skilled in the art, medical instruments need to be sterilized before they are used on a patient before a surgical operation. Sterilized instruments can come in the form of new instruments opened from a sealed package right before a surgical operation or by performing a rigorous sterilization procedure on a contaminated medical instrument. After instruments become unsterilized via contact with the ground, heavy usage in a surgical operation, etc., it is impractical to carry out one of these sterilization procedures to restore a medical instrument for use in the same operation, given that many sterilization techniques can take 2 to 3 hours or more.

Flash sterilization, on the other hand, takes mere minutes to sterilize a medical instrument, but also suffers from an impracticality. Steam introduced to the instrument during this technique must be heated at high temperatures, typically between 260 and 300 degrees fahrenheit, and kept at a higher pressures of around 20-30 pounds per square inch. If one wishes to flash sterilize a medical instrument which had become contaminated during an operation, that instrument would need to be transported out of the operation room, into a flash sterilization chamber, and carefully transported back to the operation room. Machinery and devices necessary to carry out flash sterilization may not be present in the hospital, and even then it is common for patients/doctors to burn themselves on instruments freshly sterilized with this procedure. The common practice, in place of sterilizing surgical instruments directly, is to stockpile pre-sterilized instruments before a surgical operation and hope that the supply of instruments available is enough to get through the surgical operation.

As such, it can be seen that improved disinfectant and sterilization solutions and methods are needed which both effectively clean and/or sterilize an instrument while also mitigating the effects of corrosion on the medical instruments they are used to treat.

### BRIEF SUMMARY

To solve these and other problems, compositions and methods of using those compositions are disclosed which may clean/sterilize an instrument while minimizing the corrosive damage to these instruments. These compositions may comprise an anticorrosive agent and an antiseptic. Specifically, the combination of an anticorrosive agent, particularly methylene blue, and an antiseptic, such as chlorhexidine, hydrogen peroxide, povidone iodine, sodium hypochlorite, citric acid, isopropyl alcohol, or combinations thereof, has been found to work synergistically to give effectual cleaning/sterilization results while minimizing the effects of corrosion on an instrument the composition is used upon. The antiseptic may be operative to kill and/or hinder the growth of microbes on the instrument. The composition may comprise additional components, including, but not being limited to, an antibiotic, an indicator component, an adjuvant, and/or a tracer solution. These additional components may serve to increase the cleaning functionality of the composition and/or provide additional information to a user like acting as indicator elements.

The composition may take a solid or liquid form. The composition could be formulated as a solution, which may be formed via a solid/liquid containing one or more of the components of the composition being dissolved in a solvent such as sterilized water. The composition may be applied to the instrument in numerous ways, including spraying the composition onto the instrument and/or submerging at least a portion of the instrument in the solution, although the entire instrument may be submerged in the solution in other embodiments. The compositions have been shown to be effective at cleaning/sterilizing medical instruments used in a surgical operation, although it is contemplated that these compositions can be used in other endeavors outside of the medical field. It has been found that these compositions may yield noticeably potent results when cleaning instruments via the performance of a treatment methodology, while only requiring a relatively short time-scale to achieve that potency. As such, these compositions may prove useful in cleaning/sterilizing medical instruments used intraoperatively in surgical operations via applying a composition to the medical instrument in between the surgical steps that may utilize said medical instrument.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a first step of an experimental setup, in which surgical instruments are exposed to contamination;

FIG. 2 is a second step of an experimental setup, in which surgical instruments are submerged in a composition of this present disclosure;

FIG. 3 shows the results of a first trial of an experiment testing the effectiveness of various compositions according to this present disclosure;

FIG. 4 shows the results of a second trial of an experiment testing the effectiveness of various compositions according to this present disclosure;

FIG. 5 shows the results of a third trial of an experiment testing the effectiveness of various compositions according to this present disclosure; and

FIG. 6 shows the results of a fourth trial of an experiment testing the effectiveness of various compositions according to this present disclosure.

Common reference numerals are used throughout the drawings and the detailed description to indicate the same elements.

### DETAILED DESCRIPTION

Contemplated herein are compositions and methods of using such compositions to clean/sterilize an instrument. The compositions may comprise an anticorrosive agent and an antiseptic. The anticorrosive agent may be operative to substantially reduce the corrosive effects the antiseptic(s) may otherwise impart on an instrument the composition is used upon, and the antiseptic(s) may be operative to kill and/or impede the growth of microbial contaminants, including bacteria, yeast, archaea, protozoa, fungi, algae, molds, lichen, viruses, and/or prions. In particular, a composition comprising an anticorrosive agent of methylene blue, and an antiseptic of chlorhexidine (CHG), hydrogen peroxide, povidone iodine, sodium hypochlorite, citric acid, isopropyl alcohol, or combinations thereof has been found to give synergistic, effective sterilization results with minimal corrosive effects on the instrument those compositions are used upon. It is also contemplated that the composition may further comprise an antibiotic. An example of an antibiotic that has been tested is vancomycin. Additional, optional additives the composition may further comprise include a tracer solution operative to highlight the residual bioburden on the instrument as well as an adjuvant operative to adjust the color and/or pH of the composition/a solution which incorporates the composition. One of these components and/or an additional indicator component that the composition could further include may act as an indicator element operative to react with a contaminant on the instrument, in order to indicate to a user that the particular contaminant is present. The concentrations of these components may be varied to give different levels of cleaning to an instrument they are used upon (e.g., simply killing a sufficient amount of microbes vs. full sterilization). Such compositions have been found to clean/sterilize an instrument within minutes of at least a portion of the instrument being submerged in these compositions; such compositions may therefore provide practical and effective cleaning/sterilizing procedures for medical instruments during surgical operations to prevent intraoperative contamination upon a patient.

The composition can take a solid or liquid form, both of which may be added and mixed with an existing sterile solution, such as those conventionally employed in splash basins. If the composition takes the form of a solution, it can be diluted as desired from one application to the next. It is contemplated that the composition can be implemented in intraoperative cleaning/sterilization procedure(s) for medical instruments used in a surgical operation via a number of application methods, including dissolution of the composition into an existing sterilization solution to be employed in the reservoir of a splash basin cleaning procedure, using the composition as the sole standalone sterilization solution in the reservoir of a splash basin cleaning procedure, applying the composition to the medical instrument via a sterile spray and/or a cloth soaked in the composition, and incorporating the composition into an instrument tray which may receive and hold the medical instrument. Combinations of these application methods can also be used upon the same instrument, and any particular application method can performed before or after another. It is also contemplated that the composition can be transferred in serval types of containers, including packets, syringes, and other sterile containers for case of transport and/or access prior to an operation. If sterilization is to be performed, the certain methodologies may be more effective at quickly sterilizing the instrument than others, as would be appreciated by those skilled in the art (for example, complete submersion of the instrument in a composition formulated as a solution may be more effective than partial submersion of that instrument). It also may be necessary to carry out these methodologies on longer time scales to achieve full sterilization, but the difference in time needed may only require a few extra minutes. In particular, it is contemplated that at least a portion of a medical instrument, or the entire medical instrument, can be submerged in a composition for less than a minute, at least a minute, less than two minutes, at least two minutes, less than three minutes, at least three minutes, less than four minutes, at least four minutes, less than five minutes, or at least five minutes.

While working embodiments of these compositions have been found to be effective at cleaning/sterilizing medical instruments used in surgical operations, it is contemplated that these compositions could prove useful in other fields in which it is desirous to clean/sterilize an instrument while minimizing the effects of corrosion on that instrument. Such other instruments from other technical fields may include, but are not strictly limited to, laboratory utensils used in laboratory experiments, machinery and tools used in medicine, drug, food, and/or beverage production, equipment and machines used in the manufacture and packaging of health and medical products such as toothpastes, detergents, medical dressings/compresses (including adhesive bandages), etc.

As mentioned previously, the composition may be formulated as a solution with diluted concentrations of the components present therein. The percentage values of certain components in a solution, as used throughout this present disclosure, are calculated as weight of the component over the volume of the solution. It is contemplated that these concentrations can be adjusted as needed from one application to the next, as certain cleaning/sterilization procedures may require a higher concentration of certain components, particularly the antiseptic(s), to achieve greater cleaning/sterilizing results. Effective working embodiments of a composition formulated as a solution may comprise 0.0001-10% anticorrosive agent and 0.001%-10% antiseptic. In more preferred embodiments, the solution can comprise 0.01%-10% anticorrosive agent and 0.05%-5% antiseptic. In even more preferred embodiments, the solution could be comprised of 0.005%-0.20% anticorrosive agent with 1%-4% antiseptic. In the most preferred embodiment, the solution may comprise 0.009%-0.11% anticorrosive agent and 2.5%-3.5% antiseptic. The above antiseptic concentrations may be the total concentration of all antiseptics in a solution, and as such individual antiseptics may be present at respective concentrations which differ from one another, with some specific examples of this scenario being described below.

Ideally, the anticorrosive agent would be methylene blue, which has been found to mitigate the effects of corrosion that antiseptics or other additives may otherwise impart on a surgical instrument. In addition to the anticorrosive agent concentrations listed above, it is contemplated that methylene blue may be present in a composition at more dilute concentrations. Specifically, these compositions may comprise 0.001%-0.1% methylene blue, more preferably 0.005%-0.05% methylene blue, even more preferably 0.007%-0.03% methylene blue, and most preferably 0.009%-0.011% methylene blue. The antiseptic may include a combination of chlorohexidine and hydrogen peroxide, although alternative/additional antiseptics may be present in a composition, including povidone iodine, sodium hypochlorite, isopropyl alcohol, and/or citric acid. A composition formulated as a solution could comprise 0.01%-5% chlorohexidine, more preferably 0.02%-1% chlorohexidine, even more preferably 0.03%-0.5% chlorohexidine, and most preferably 0.04%-0.06% chlorohexidine. Higher concentrations of chlorohexidine may be more suitable for applications involving infected prosthesis or soiled/contaminated wounds. As for hydrogen peroxide, a composition formulated as a solution could comprise 0.01%-5% hydrogen peroxide, more preferably 0.5%-4.5% hydrogen peroxide, even more preferably 1%-4% hydrogen peroxide, and most preferably 2.5%-3.5% hydrogen peroxide.

Working embodiments have also been tested with antibiotics. A composition formulated as a solution may comprise 0.01-10% antibiotic, more preferably 0.02-5% antibiotic, even more preferably 0.05%-1% antibiotic, and most preferably, and 0.09%-0.11% antibiotic. Specifically, vancomycin was tested, as will be shown later in this disclosure, but other antibiotics can be used other than or in addition to vancomycin as needed for a particular situation and/or type of bacterial coverage desired. For example, a general surgery case may use an antibiotic with more gram-negative coverage, while a orthopedics case may use an antibiotic with better gram positive/MRSA (Methicillin-resistant Staphylococcus aureus) coverage. Suitable antibiotics include, but are not limited to, penicillin, tetracycline, sulfonamide, carbapenem, nitrofurantoin, rifamycin, delafloxacin, aminoglycoside, clindamycin, trimethoprim, tigecycline, imipenem, macrolides, cephalosporin, lefamulin, daptomycin, cephalexin, doxycycline, vancomycin, gentamycin, tobramycin, cefazolin, teicoplanin, and amoxicillin.

Indicator components may be included as an additive to a composition so that a reaction occurs if a particular contaminant is introduced to the composition, which one may observe to recognize the presence of that contaminant. However, one of the other components of a composition, like the anticorrosive agent of antiseptic, may function as an indicator component of a composition, either alone or in addition to a separate indicator component. For example, a reaction of methylene blue with human blood may cause a composition to become blue and opaque, while a reaction of hydrogen peroxide with human blood may cause bubbling. The concentration of certain components may be adjusted to act as a stronger or weaker indicator element.

The remaining description will discuss an experimental setup that was conducted with various compositions according to this disclosure and the outcomes from those experiments, highlighting how such compositions may impede the growth of microbial contaminants on surgical instruments while leaving minimal corrosion on those instruments.

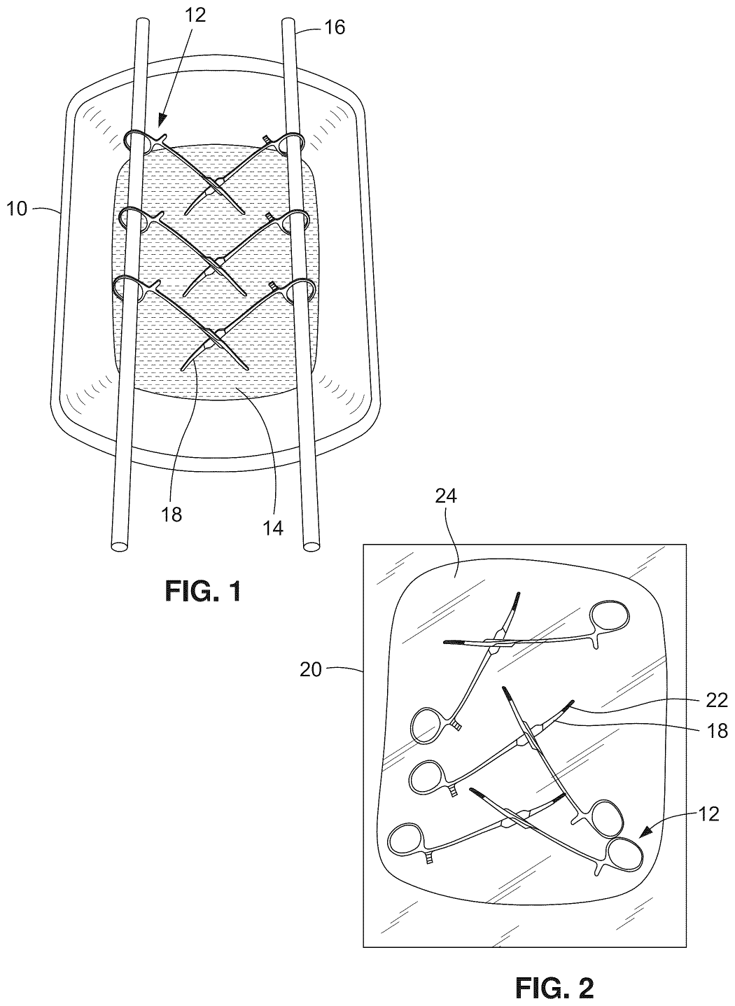

Referring now to FIG. 1, a first step of an experimental setup in which surgical instruments are exposed to contamination is shown. An MSRA strain was placed in a 3 mL saline tube with a concentration ranging from 0.5 to 1.0 McFarland. This inoculum was added to 1 L of whole blood in a plastic bin 10 at room temperature and allowed to sit for thirty minutes. After this time passed, the tips 18 of hemostats 12 were partially submerged in the blood solution 14 for five minutes. The experimental setup, in which the hemostats 12 were suspended by supports 16, ensured that the tips 18 of each hemostat 12 were submerged at the same standardized depth.

Turning now to FIG. 2, a second step of an experimental setup, in which surgical instruments are submerged in a composition of this present disclosure, is depicted. After the five minutes had passed after partial submersion in the blood solution 14, the hemostats 12 were removed from the first plastic bin 10 and placed in a second, plastic bin 20, where they were allowed to dry for twenty minutes. Following this drying of the hemostats 12, a composition 24 formulated as a solution in sterile water was introduced to the bin 20. Enough of the composition 24 was added to the bin 20 so that the hemostats 12 were completely submerged in the composition 24. The hemostats 12 were allowed to sit completely submerged in the composition 24 for one minute. A control group of hemostats was not submerged in a composition 24 and instead sent to the next step following drying in the bin 20. Four trials were performed, with each trial testing a certain set of compositions 24 on three hemostats 12, as will be detailed below in relation to the remaining figures of this disclosure.

After submerging the non-control group hemostats 12 in their assigned compositions 24, the hemostats 12 were removed from the plastic bin 20. No visible corrosion was appreciated on all hemostats 12 tested. The hemostats 12 were then placed in ten cc tubes of tryptic soy broth. The broth solution was vortexed until no visible blood remained on the hemostats 12. The tryptic soy broth, now carrying the blood from the hemostats 12, was cultured onto blood agar plates. The number of colonies forming units on the plates after 2-3 days was then measured and compared to see the effects the compositions 24 had on inhibiting the growth of these colonies.

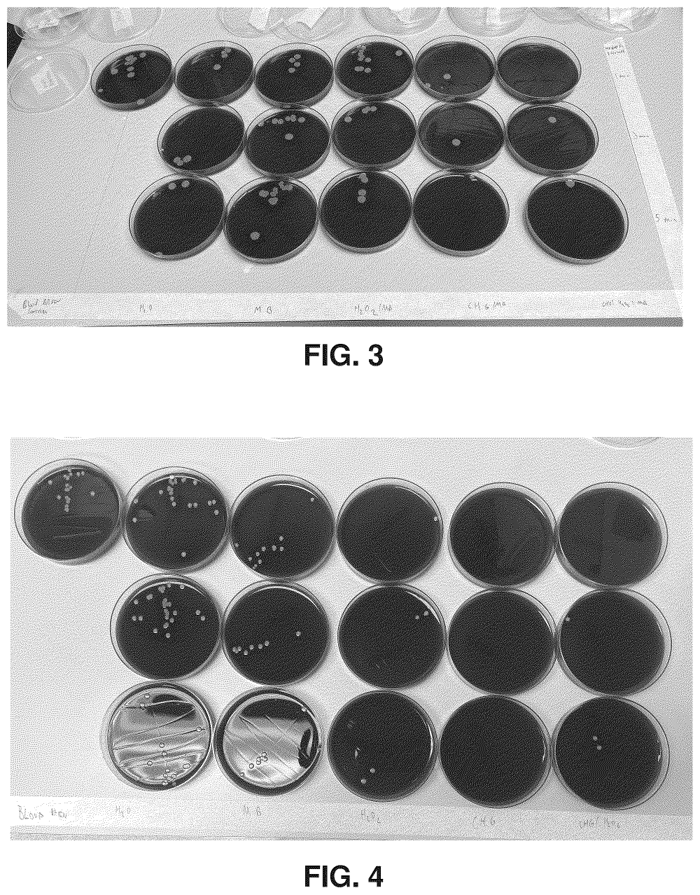

Referring now to FIGS. 3 and 4, the results of a first and second trial respectively of experiments testing the effectiveness of various compositions according to this present disclosure are shown. The set of compositions tested in both the first and second trials are listed in Table 1 below.

TABLE 1

| | CONCENTRATION OF RELEVANT |
|---|---|
| SOLUTION NAME | COMPONENTS |
| Water | N/A (sterile water) |
| Methylene Blue | 0.1% methylene blue |
| Methylene Blue + Hydrogen Peroxide | 0.1% methylene blue, 3% hydrogen peroxide |
| Methylene Blue + Chlorohexidine | 0.1% methylene blue, 0.05% chlorohexidine |
| Methylene Blue + Hydrogen Peroxide + | 0.1% methylene blue, 3% hydrogen peroxide, |
| Chlorohexidine | 0.05% chlorohexidine |

The results of the first trial are shown in FIG. 3. The left-most plate corresponds to a control group hemostat which was not submerged in a composition. Each preceding column of agar plates to the right corresponds to the three hemostats submerged in one of the solutions above from Table 1; specifically, from left to right, the columns of 3 plates correspond to the “Water”, “Methylene Blue”, “Methylene Blue+Hydrogen Peroxide”, “Methylene Blue+Chlorohexidine”, and “Methylene Blue+Hydrogen Peroxide+Chlorohexidine” solutions in Table 1. The number of colonies that formed on these plates three days after culturing is described in Table 2 below, with the left-most column of Table 2 referring to the names of solutions defined in Table 1. The plates are numbered 1-3 from top to bottom, such that plate 1 corresponds to the plate at the top of the respective column shown in the figure.

TABLE 2

| | Number of | Number of | Number of | Average |
|---|---|---|---|---|
| SOLUTION | Colonies on | Colonies on | Colonies on | Number of |
| TESTED | Plate 1 | Plate 2 | Plate 3 | Colonies |
| N/A (Control) | 13 | N/A | N/A | 13 |
| Water | 5 | 3 | 2 | 3.3 |
| Methylene Blue | 4 | 8 | 17 | 9.7 |
| Methylene Blue + | 13 | 4 | 4 | 7 |
| Hydrogen | | | | |
| Peroxide | | | | |
| Methylene Blue + | 3 | 1 | 0 | 1.3 |
| Chlorhexidine | | | | |
| Methylene Blue + | 0 | 1 | 1 | 0.7 |
| Hydrogen | | | | |
| Peroxide + | | | | |
| Chlorhexidine | | | | |

The results of the second trial are shown in FIG. 4. The same arrangement of plates and how they correspond to the compositions listed in Table 1 above applies here. Table 3 below describes the number of colonies which formed on these plates after two days of culturing, with the left-most column of Table 3 referring to the names of solutions defined in Table 1. The plates are numbered 1-3 from top to bottom, such that plate 1 corresponds to the plate at the top of the respective column shown in the figure.

TABLE 3

| | Number of | Number of | Number of | Average |
|---|---|---|---|---|
| | Colonies on | Colonies on | Colonies on | Number of |
| Solution Used | Plate 1 | Plate 2 | Plate 3 | Colonies |
| N/A (Control) | 19 | X | X | 19 |
| Water | 24 | 28 | 20 | 24 |
| Methylene Blue | 11 | 8 | 9 | 9.3 |
| Methylene Blue + | 1 | 2 | 2 | 1.7 |
| Hydrogen | | | | |
| Peroxide | | | | |
| Methylene Blue + | 0 | 0 | 0 | 0 |
| Chlorhexidine | | | | |
| Methylene Blue + | 0 | 1 | 2 | 1 |
| Hydrogen | | | | |
| Peroxide + | | | | |
| Chlorhexidine | | | | |

It can be seen that the compositions comprising both methylene blue and chlorhexidine clearly show a noticeable improvement when compared to the control and water groups. In these trials, the effectiveness of a methylene blue and hydrogen peroxide solution is also made apparent.

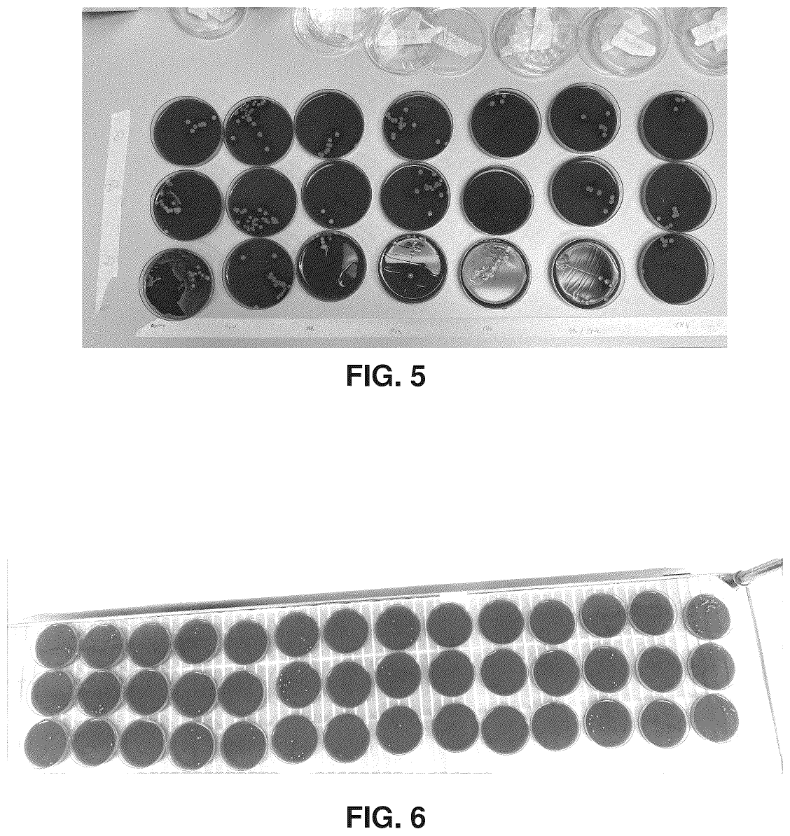

Referring now to FIG. 5, the results of a third trial of an experiment testing the effectiveness of various compositions according to this present disclosure are shown. The third trial differs when compared to the first two trials in that a total of three control hemostats, resulting in three control agar plates, were tested, and that a new composition was also tested which included the antibiotic of vancomycin. Table 4 below lists the compositions that were tested in this third trial.

TABLE 4

| | CONCENTRATION OF RELEVANT |
|---|---|
| SOLUTION NAME | COMPONENTS |
| Water | N/A (just sterile water) |
| Methylene Blue | 0.1% methylene blue |
| Methylene Blue + Hydrogen Peroxide | 0.1% methylene blue, 3% hydrogen peroxide |
| Methylene Blue + Chlorohexidine | 0.1% methylene blue, 0.05% chlorohexidine |
| Methylene Blue + Hydrogen Peroxide + | 0.1% methylene blue, 3% hydrogen peroxide, |
| Chlorohexidine | 0.05% chlorohexidine |
| Methylene Blue + Hydrogen Peroxide + | 0.1% methylene blue, 3% hydrogen peroxide, |
| Chlorohexidine + Vancomycin | 0.05% chlorohexidine, 1% vancomycin |

The left-most column of three plates in FIG. 5 correspond to a control group of hemostats which were not submerged in a composition. Each preceding column of agar plates corresponds to the three hemostats submerged in one of the solutions defined in Table 4 above. Specifically, from left to right, the columns of 3 plates after the first column correspond to the “Water”, “Methylene Blue”, “Methylene Blue+Hydrogen Peroxide”, “Methylene Blue+Chlorohexidine”, “Methylene Blue+Hydrogen Peroxide+Chlorohexidine”, and “Methylene Blue+Hydrogen Peroxide+Chlorohexidine+Vancomycin” compositions defined in Table 4. The number of colonies that formed on these plates three days after culturing is described in Table 5 below. The plates are numbered 1-3 from top to bottom, such that plate 1 corresponds to the plate at the top of the respective column shown in the figure.

TABLE 5

| | Number of | Number of | Number of | Average |
|---|---|---|---|---|
| SOLUTION | Colonies on | Colonies on | Colonies on | Number of |
| TESTED | Plate 1 | Plate 2 | Plate 3 | Colonies |
| N/A (Control) | 7 | 17 | 14 | 12.7 |
| Water | 48 | 34 | 24 | 35.7 |
| Methylene Blue | 13 | 2 | 6 | 7 |
| Methylene Blue + | 19 | 17 | 13 | 16.3 |
| Hydrogen | | | | |
| Peroxide | | | | |
| Methylene Blue + | 4 | 0 | 4 | 2.7 |
| Chlorhexidine | | | | |
| Methylene Blue + | 6 | 6 | 5 | 5.7 |
| Hydrogen | | | | |
| Peroxide + | | | | |
| Chlorhexidine | | | | |
| Methylene Blue + | 5 | 7 | 6 | 6 |
| Hydrogen | | | | |
| Peroxide + | | | | |
| Chlorhexidine + | | | | |
| Vancomycin | | | | |

The methylene blue+chlorhexidine solutions are once again shown here to give a noticeable improvement at preventing the growth of microbial colonies on the agar plates.

Referring now to FIG. 6, the results of an experiment testing the effectiveness of various compositions according to this present disclosure in a fourth trial are shown. The compositions listed in Table 6 below were tested.

TABLE 6

| | CONCENTRATION OF RELEVANT |
|---|---|
| SOLUTION NAME | COMPONENTS |
| Water | N/A (just sterile water) |
| Methylene Blue | 0.1% methylene blue |
| Methylene Blue + High Hydrogen Peroxide | 0.1% methylene blue, 3% hydrogen peroxide |
| Methylene Blue + Low Chlorohexidine | 0.1% methylene blue, 0.05% chlorohexidine |
| Methylene Blue + High Chlorohexidine | 0.1% methylene blue, 0.5% chlorohexidine |
| Methylene Blue + Povidone Idoine | 0.1% methylene blue, 0.3% povidone iodine |
| Methylene Blue + Sodium Hypochlorite | 0.1% methylene blue, 0.5% sodium hypochlorite |
| Methylene Blue + Low Hydrogen Peroxide + Low | 0.1% methylene blue, 0.1% hydrogen peroxide, |
| Chlorohexidine | 0.05% chlorohexidine |
| Methylene Blue + Low Hydrogen Peroxide + High | 0.1% methylene blue, 0.1% hydrogen peroxide, |
| Chlorohexidine | 0.05% chlorohexidine |
| Methylene Blue + High Hydrogen Peroxide + Low | 0.1% methylene blue, 3% hydrogen peroxide, |
| Chlorohexidine | 0.05% chlorohexidine |
| Methylene Blue + High Hydrogen Peroxide + High | 0.1% methylene blue, 3% hydrogen peroxide, |
| Chlorohexidine | 0.05% chlorohexidine |
| Methylene Blue + Low Hydrogen Peroxide + | 0.1% methylene blue, 0.1% hydrogen peroxide, |
| Povidone Iodine | 0.3% povidone iodine |
| Methylene Blue + High Hydrogen Peroxide + | 0.1% methylene blue, 3% hydrogen peroxide, |
| Povidone Iodine | 0.3% povidone iodine |

From left to right, each column of three agar plates in FIG. 6 corresponds to the “Water”, “Methylene Blue”, “Methylene Blue+High Hydrogen Peroxide”, “Methylene Blue+Low Chlorohexidine”, “Methylene Blue+High Chlorohexidine”, “Methylene Blue+Povidone lodine”, “Methylene Blue+Sodium Hypochlorite”, “Methylene Blue+Low Hydrogen Peroxide+Low Chlorohexidine”, “Methylene Blue+Low Hydrogen Peroxide+High Chlorohexidine”, “Methylene Blue+High Hydrogen Peroxide+Low Chlorohexidine”, “Methylene Blue+High Hydrogen Peroxide+High Chlorohexidine”, “Methylene Blue+Low Hydrogen Peroxide+Povidone Iodine”, and “Methylene Blue+High Hydrogen Peroxide+Povidone Iodine.” The last column of agar plates (on the far right of FIG. 6) corresponds to a negative control group of hemostats which were not submerged in a composition. Table 7 below describes the number of colonies that formed on these agar plates 3 days after culturing. The plates are numbered 1-3 from top to bottom, such that plate 1 corresponds to the plate at the top of the respective column shown in the figure.

TABLE 7

| | Number of | Number of | Number of | Average |
|---|---|---|---|---|
| SOLUTION | Colonies on | Colonies on | Colonies on | Number of |
| TESTED | Plate 1 | Plate 2 | Plate 3 | Colonies |
| Water | 5 | 2 | 2 | 2 |
| Methylene Blue | 5 | 7 | 1 | 4.3 |
| Methylene Blue + | 1 | 0 | 0 | 0.3 |
| [Low/High] | | | | |
| Hydrogen | | | | |
| Peroxide | | | | |
| Methylene Blue + | 2 | 2 | 4 | 2.7 |
| Low | | | | |
| Chlorohexidine | | | | |
| Methylene Blue + | 0 | 0 | 2 | 0.7 |
| High | | | | |
| Chlorohexidine | | | | |
| Methylene Blue + | 4 | 10 | 5 | 6.3 |
| Povidone Idoine | | | | |
| Methylene Blue + | 1 | 0 | 2 | 1 |
| Sodium | | | | |
| Hypochlorite | | | | |
| Methylene Blue + | 3 | 2 | 2 | 2.3 |
| Low Hydrogen | | | | |
| Peroxide + Low | | | | |
| Chlorohexidine | | | | |
| Methylene Blue + | 0 | 0 | 0 | 0 |
| Low Hydrogen | | | | |
| Peroxide + High | | | | |
| Chlorohexidine | | | | |
| Methylene Blue + | 0 | 0 | 0 | 0 |
| High Hydrogen | | | | |
| Peroxide + Low | | | | |
| Chlorohexidine | | | | |
| Methylene Blue + | 0 | 0 | 0 | 0 |
| High Hydrogen | | | | |
| Peroxide + High | | | | |
| Chlorohexidine | | | | |
| Methylene Blue + | 1 | 6 | 6 | 4.3 |
| Low Hydrogen | | | | |
| Peroxide + | | | | |
| Povidone Iodine | | | | |
| Methylene Blue + | 0 | 0 | 1 | 0.3 |
| High Hydrogen | | | | |
| Peroxide + | | | | |
| Povidone Iodine | | | | |
| N/A (Control) | 25 | 2 | 7 | 11.3 |

From these results, it can be appreciated that the combination of chlorohexidine and hydrogen peroxide was the most effective at mitigating bacterial growth. Different concentration combinations of these antiseptics were proven to be successful. The effectiveness of other antiseptics, such as povidone iodine and sodium hypochlorite, can also be recognized.

The above description is given by way of example, and not limitation. Given the above disclosure, one skilled in the art could devise variations that are within the scope and spirit of the invention disclosed herein, including various ways of formulating these compositions and various methods of implementing those compositions. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A composition for cleaning an instrument while mitigating the effects of corrosion on the instrument, the composition comprising:
an anticorrosive agent in a range of 0.001% to 10% by weight over volume of the composition, and
an antiseptic in a range of 0.001% to 10% by weight over volume of the composition,
wherein the anticorrosive agent is methylene blue.

2. The composition of claim 1, wherein the composition comprises the anticorrosive agent in a range of 0.001% to 1% by weight over volume of the composition.

3. The composition of claim 1, wherein the antiseptic comprises hydrogen peroxide, chlorohexidine, povidone iodine, sodium hypochlorite, isopropyl alcohol, citric acid, or combinations thereof.

4. The composition of claim 1, wherein the antiseptic comprises 0.001%-5% chlorohexidine by weight over volume of the composition and 0.01%-5% hydrogen peroxide by weight over volume of the composition.

5. The composition of claim 1, wherein the composition further comprises an antibiotic.

6. The composition of claim 1, wherein the instrument is a medical instrument used during a surgical operation.

7. The composition of claim 1, wherein the composition is formulated as a solution dissolved in a solvent.

8. The composition of claim 7, wherein the solvent is sterilized water.

9. The composition of claim 1, wherein the composition is a solid.

10. A method for cleaning an instrument while mitigating the effects of corrosion on the instrument, the method comprising the step of:
applying a composition to the instrument;
wherein the composition comprises an anticorrosive agent and an antiseptic; and
wherein the anticorrosive agent is methylene blue.

11. The method of claim 10, wherein the composition is formulated as a solution dissolved in a solvent.

12. The method of claim 11, wherein the step of applying the composition is performed via spraying the composition onto the instrument, submerging at least a portion of the instrument in the solution, or a combination thereof.

13. The method of claim 12, wherein during the submerging at least a portion of the instrument in the solution, the portion of the instrument is submerged in the solution for at least one minute.

14. The method of claim 12, wherein the entire instrument is submerged in the solution during the submerging of at least the portion of the instrument in the solution.

15. The method of claim 11, the method further comprising a step of forming the solution, wherein the solution is formed via mixing the solvent with a solid comprising the anticorrosive agent and the antiseptic.

16. The method of claim 10, wherein the composition comprises methylene blue in a range of 0.001 to 10% by weight over volume of the composition.

17. The method of claim 10, wherein the composition comprises antiseptic in a range of 0.001 to 10% by weight over volume of the composition.

18. The method of claim 10, wherein the instrument is a medical instrument used during a surgical operation.

19. The method of claim 10, wherein the composition further comprises an antibiotic.

20. The method of claim 10, wherein the antiseptic comprises hydrogen peroxide, chlorohexidine, povidone iodine, sodium hypochlorite, isopropyl alcohol, citric acid, or combinations thereof.
